(19) United States
(12) Patent Application Publication
(10) Pub. No.: US 2017/0097325 A1
(43) Pub. Date: Apr. 6, 2017

(54) DEVICE AND METHOD FOR EXTRACTION OF COMPOUNDS WITH AROMATIC CYCLES CONTAINED IN A LIQUID SAMPLE

(71) Applicant: Commissariat a L'Energie Atomique et aux Energies Alternatives, Paris (FR)

(72) Inventors: Louise FOAN, Castanet Tolosan (FR); Severine VIGNOUD, Bernin (FR); Florence RICOUL, Quaix-En-Chartreuse (FR)

(73) Assignee: Commissariat a L'Energie Atomique et aux Energies Alternatives, Paris (FR)

(21) Appl. No.: 15/280,227

(22) Filed: Sep. 29, 2016

(30) Foreign Application Priority Data
Oct. 2, 2015 (FR) 15 59405

(51) Int. Cl. B01J 20/281; C01B 31/30; B01J 20/02; G01N 30/06; B01J 20/28; B01J 20/32; C07C 7/10; G01N 30/60; B01D 15/22; B01J 20/282

(52) U.S. Cl. CPC G01N 30/482; B01D 15/22; C01B 31/301; B01J 20/02; B01J 20/282; B01J 20/2808; B01J 20/28083; B01J 20/3234; B01J 20/3214; C07C 7/10; G01N 30/6095; G01N 30/06; B01J 2220/54; G01N 2030/027

(57) ABSTRACT

A device for extraction of analytes with aromatic cycles, preferably analytes with aromatic cycles for which the octanol-water partition coefficient is more than 10³, the analytes being contained in a liquid phase, the extraction device including a support and an adsorption layer at least partially covering the support, the adsorption layer being porous SiOxCyHz.

### TECHNICAL DOMAIN AND PRIOR ART

This invention relates to a device and a method for the extraction of analytes contained in a liquid ready for analysis, the analytes having one or more aromatic cycles, such as polycyclic aromatic hydrocarbons (PAH).

“Analytes” are organic compounds present in a liquid matrix.

Water quality is influenced by the presence of pollutants originating from industrial and domestic waste, and from transport and agricultural activities. The origin of contamination may be chemical (inorganic or organic pollutants) or microbiological (viruses, bacteria, parasites, etc.). Chemical contaminants include macropollutants such as nitrates and phosphates present in the aquatic environment in significant concentrations of the order of 1 mg/l, micropollutants, a term that includes metallic trace elements (or heavy metals) and organic trace compounds (PAH, pesticides, pharmaceutical substances, etc.), characterised by toxicity even at trace concentrations (between 1 ng/l and 1 μg/l). At the present time, aquatic ecosystems are monitored principally by means of laboratory analyses that require a large human and financial investment. Sampling, transport and storage steps are particularly difficult and can introduce a bias on the results obtained due to losses or contamination.

PAHs are persistent organic pollutants emitted by multiple sources (automobile traffic, industries, domestic heating) and form a serious sanitary challenge. Some are considered to be or are suspected of being carcinogenic and are therefore monitored in water.

Laboratory analysis techniques adapted to the analysis of PAHs are gas phase chromatography and high performance liquid chromatography associated with mass spectrometry and fluorimetry/UV detection, respectively.

Before samples are introduced into the analytic device, a sample treatment procedure is necessary to transfer analytes of interest from the original medium to an appropriate form ready for the analysis. Treatment of the sample also provides a means of concentrating targeted compounds and separating any interfering molecules present in the matrix.

The most frequently used techniques for the extraction of PAHs contained in water are Liquid-Liquid Extraction, Solid Phase Extraction (SPE), Solid Phase Microextraction (SPME) and Stir-Bar Sorptive Extraction (SBSE). Liquid-liquid extraction has the advantage of being easy to implement, especially if there is an automated stirring system, and it only requires a short optimisation period. However, this technique requires rigorous cleaning of the glasswork to prevent contamination of the samples. It also consumes large solvent volumes of the order of 25 ml to 300 ml per sample, and these solvents are expensive to purchase and to treat after use. Finally, liquid-liquid extraction is only possible on large sample volumes, for example between 250 ml and 1 l.

SPE, SPME and SBSE consist of immersing a solid phase containing a stationary phase with an affinity with analytes of interest, that are adsorbed onto this phase, into a liquid or a gas. The solid phase is removed from the liquid or the gas and analytes are desorbed so that they can be analysed. These techniques can reduce the consumption of solvents, for example from 6 ml to 15 ml per sample, while maintaining good extraction efficiencies and low detection limits. SPE on disks can also accelerate the sample treatment procedure. However, unlike SPME and SBSE, SPE cannot be applied to small volume samples. Furthermore, these techniques only consume small quantities of solvents. When conditioning and desorption are done by thermodesorption, the consumption of solvent is even zero. On the other hand, the yield of SPME for the analysis of PAHs is too low and quantification limits are too high, while the results obtained with SBSE are very satisfactory.

SBSE is based on extraction by sorption of molecules dissolved in the aqueous phase by a magnetic bar coated with polydimethylsiloxane (PDMS).

Compounds targeted by this technique are compounds with octanol/water partition coefficients of more than 1000 (log K_{ow}>3), which is the case for the 16 PAHs classified as priority by the United States Environmental Protection Agency (USEPA).

A thermodesorption step is then carried out, for example by inserting the SBSE bar in a stainless steel pipe on which the desorption step is done. The analytes are recondensed on a cold trap before being sent by fast heating to an analysis device, for example a gas phase chromatography column associated with a detector.

The SBSE extraction technique making use of PDMS requires a time of the order of 1 to 2 hours for PAHs with low molar masses (i.e. with 2-3 aromatic cycles), but higher mass PAHs (i.e. with 4-6 aromatic cycles) it requires a much longer time, for example more than 12 hours.

Miniaturisation and automation of laboratory extraction techniques have been undertaken in order to reduce quantities of samples and solvents used, and human working time.

Concerning field techniques, at the moment there are two types of equipment marketed to analyse PAHs in water:

UV fluorescence spectroscopy probes (Aqua MS®, CONTROS®, HACH-Lange®) for which the measurement is not specific, in other words these instruments measure the concentration of all aromatic compounds that are fluorescent at a given wavelength (λ_{excitation}=254 nm and λₑₘᵢₛₛᵢₒₙ=360 nm).

A portable Gas Chromatography combined to a Mass Spectrometry (GC-MS) (HAPSITE marketed by Inficon®) provided with an SPME sampling system that can only measure PAHs with 2-3 cycles because with extraction by SPME, it is impossible to preconcentrate PAHs with a higher molar mass with sufficient yields for the technique to be sensitive.

Document L. Foan et al. “A novel microfluidic device for fast extraction of polycyclic aromatic hydrocarbons (PAHs) from environmental waters—comparison with stir-bar sorptive extraction (SBSE)”, International Journal of Environmental Analytical Chemistry, vol. 95, n° 13, 4 Feb. 2015 discloses an extraction device for extracting PAHs contained in water; it comprises a chamber functionalized with a layer of PDMS. The extraction yields with this device for most of the PAHs are low.

### PRESENTATION OF THE INVENTION

Consequently, one purpose of this invention is to provide an extraction device that has a strong affinity with organic compounds such as PAHs.

The purpose described above is achieved using a device for extraction of analytes of interest in the liquid phase comprising at least one support covered with an adsorption layer, said layer being SiOₓC_{y}H_{z} (silicon oxycarbide) that extracts compounds such as PAHs contained in a liquid matrix.

The inventors have discovered that SiOₓC_{y}H_{z} is particularly efficient for extracting PAHs from a liquid medium, and more generally for extracting compounds with aromatic cycles, and particularly compounds with aromatic cycles with an octanol-water partition coefficient of more than 10³. The latter has an affinity with these compounds that makes their extraction by SiOₓC_{y}H_{z} particularly efficient compared with other materials such as PDMS that has a limited efficiency with compounds with aromatic cycles, and particularly with compounds with a relatively low octanol/water partition coefficient, in other words between 10³ and 10⁵.

In one advantageous example, the extraction device comprises an extraction chamber the internal surfaces of which form the support, the extraction chamber comprising a supply to said liquid zone containing the analytes and an evacuation outlet from said zone. Since the liquid sample is injected into a closed chamber, the device is more practical to use than manipulation of a bar.

Furthermore, extraction is faster than with other techniques such as the SBSE technique and only requires a small sample volume. This extraction speed makes this device particularly suitable for portable use.

Advantageously, the extraction zone contains microstructures, for example it comprises micropillars coated with SiOₓC_{y}H_{z}.

Advantageously, an analysis device such as a chromatography column associated with a detector, advantageously a chromatography microcolumn associated with a detector, is connected to the outlet from the extraction device, compounds adsorbed by SiOₓC_{y}H_{z} being desorbed before being sent to the chromatography column. The extraction speed and integration of the extraction device and the analysis device make it possible to obtain analysis results quickly and directly on the sample taking zone.

The subject-matter of this invention is then a device for the extraction of analytes with aromatic cycles, said analytes being contained in a liquid phase, said extraction device comprising a support and an adsorption layer being porous SiOxCyHz, said adsorption layer at least partially covering said support.

Very advantageously, analytes with aromatic cycles have an octanol-water partition coefficient greater than 10³, and preferably the octanol-water partition coefficient is between 10³ and 10⁵.

x may be between 1 and 2 and preferably between 1.4 and 1.8; y may be between 0.8 and 3 and preferably between 1 and 2.5 and z may be between 2.5 and 4.5 and preferably between 3 and 4.1.

The thickness of the adsorption layer may for example be between 50 nm and 2000 nm, and preferably between 50 nm and 1000 nm.

The porosity of the adsorption layer may be between 3% and 60%, and preferably between 10% and 40% and pores may have a radius of between 1 nm and 5 nm.

In one example embodiment, the support is composed of at least the walls of an extraction chamber, said extraction chamber comprising at least one liquid phase supply orifice and at least one liquid phase outlet orifice, said supply and outlet orifices being arranged such that the liquid phase comes into contact with the adsorption layer and flows from the supply orifice towards the extraction orifice.

Preferably, the extraction chamber comprises microstructures at least partly coated by the adsorption layer composed of porous SiOxCyHz. The microstructures may be micropillars supporting the absorption layer.

The device may include a box containing the chamber, and a cover closing the chamber.

Another subject-matter of this invention is an analysis system comprising an extraction device according to the invention and a device for the analysis of compounds extracted by the extraction device.

The analysis device may be a chromatography column associated with a detector, advantageously a chromatography microcolumn associated with a detector.

Another subject-matter of this invention is a method of manufacturing an extraction device according to the invention, including the following steps:
a) make a support,
b) form a layer of SiOxCyHz on the support,

Step b) may be done by chemical vapour deposition, advantageously plasma-enhanced chemical vapour deposition.

A porogen, for example norbornadiene, can be used in step b) during formation of the SiOxCyHz layer, with an annealing step to eliminate the porogen.

The support may be formed by the internal walls of an extraction chamber, said chamber being made by photolithography and etching, advantageously DRIE etching.

Another subject-matter of this invention is a method for extraction of compounds with aromatic cycles that advantageously have octanol/water partition coefficients of more than 10³ contained in a liquid phase, making use of an extraction device according to the invention including the following steps:
bring a liquid sample comprising at least one aromatic compound advantageously having an octanol/water partition coefficient of more than 10³ into contact with the absorption layer

### BRIEF DESCRIPTION OF THE DRAWINGS

This invention will be better understood after reading the following description and drawings on which:

FIG. 1 is a diagrammatic view of an example of an extraction device according to the invention,

FIGS. 2A and 2B are images of the inside of an example extraction device in FIG. 1 obtained by a scanning electron microscope with two different magnifications,

FIG. 3 is a graphic view of the extraction yield obtained by an extraction device according to the invention and using the SBSE technique and a magnetic bar,

FIG. 4 shows a graphic view of adsorption isotherms of different PAHs representing the variation of the mass of different PAHs adsorbed by mass per phase as a function of the initial concentration in the studied solution in μg/L with an extraction device making use of a PDMS layer (curves in dashed lines) and with an extraction device according to the invention using an SiOxCyHz layer (curves in solid lines).

FIG. 5 is a graphic view of the distribution of pesticides as a function of the value of their K_{ow},

FIG. 6 is a table containing several pharmaceutical substances with the value of their log K_{ow}.

### DETAILED PRESENTATION OF PARTICULAR EMBODIMENTS

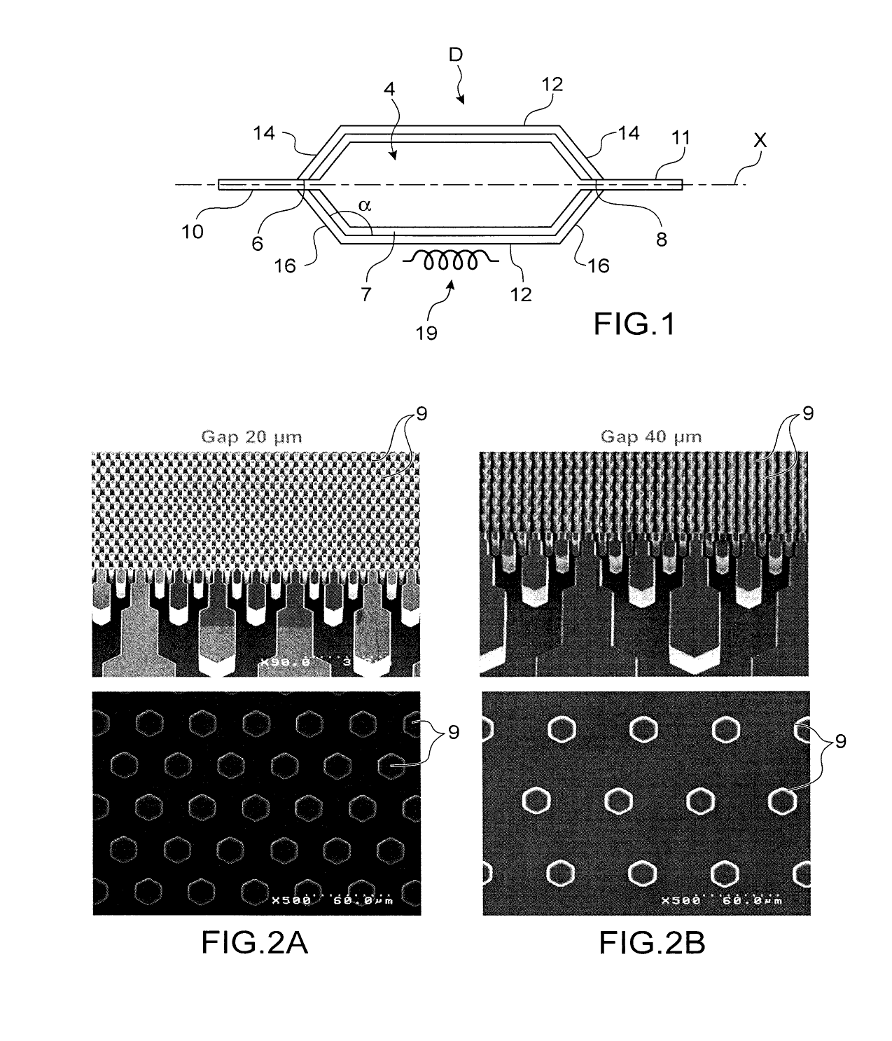

FIG. 1 shows a diagrammatic view of an example embodiment of an extraction device according to the invention,

The device D comprises an extraction zone by absorption and desorption 4 extending along a longitudinal axis X. The zone 4 comprises a supply orifice 6 and an outlet orifice 8 for supplying the extraction zone 4 with fluid and for evacuation from the extraction zone 4, respectively.

For reasons of simplicity, the extraction zone by adsorption and desorption will be referred to as “extraction zone 4” throughout the remainder of this description.

For example, the device D comprises a box and a cover, the extraction zone 4 being made in the box, and is closed and sealed by the cover

The extraction zone 4 comprises a chamber provided with a surface on the inside of which analytes will be adsorbed and absorbed. The extraction zone 4 preferably has a large surface to volume ratio so that analytes can be analysed efficiently.

The inside surface of the chamber is at least partly covered with a layer of SiOₓC_{y}H_{z} on which the compounds will be adsorbed.

In this example, the extraction zone extends in three dimensions, but it will be understood that according to the invention, the extraction zone may be a zone extending in two dimensions and covered with a layer of SiOxCyHz, for example a plate or a strip that will be dipped in a liquid containing the analytes to be adsorbed. The liquid is an aqueous solution.

x is preferably between 1 and 2 and preferably between 1.4 and 1.8; y is between 0.8 and 3 and preferably between 1 and 2.5 and z is between 2.5 and 4.5 and preferably between 3 and 4.1.

The porosity of the SiOxCyHz layer may for example be between 3% and 60%, and preferably between 10% and 40%. This percentage represents the volume of pores for a given volume of material. The radius of the pores is preferably between 1 nm and 3 nm, and even between 1 nm and 5 nm. These dimensions have been observed by ellipso-porosimetry, the probe molecule being toluene, the measuring device being the EP12 model made by the SOPRA company.

The thickness of the SiOₓC_{y}H_{z} layer is preferably between 50 nm and 2000 nm, and even more preferably between 50 nm and 1000 nm.

Advantageously, the extraction zone 4 comprises microstructures that provide a large adsorption surface area.

These microstructures preferably extend from the bottom of the chamber. These microstructures may for example be micropillars 9.

FIGS. 2A et 2B are photos of an adsorption zone containing micropillars taken with a scanning electron microscope. The gap between pillars is for example 20 μm and 40 μm on FIGS. 2A and 2B respectively, and preferably between 10 μm and 100 μm, and the transverse dimensions of the micropillars is preferably between 10 μm and 100 μm, for example equal to 20 μm.

The cross-section of the micropillars in a plane perpendicular to the centre-line of the micropillars is preferably such that it limits flow disturbances. In the example shown in FIGS. 2A and 2B, the cross-section of the micropillars is hexagonal. The piers are preferably oriented such that they are in the direction of flow symbolise by the arrow F so as to limit head losses. In the example shown, the pads have two faces approximately parallel to the flow and four inclined faces along which flow passes. As a variant, the micropillars could have a square cross-section or any other cross-section with a large surface area. This shape is also compatible with manufacturing methods making use of microelectronic techniques.

Nevertheless, it will be understood that the micropillars could have a cross-section with a different shape, for example square or circular, without going outside the framework of this invention.

The adsorption layer 7 is carried by microstructures and by the surface of the chamber walls. These microstructures increase the surface area on which analytes can be adsorbed, which increases the sensitivity of the analysis.

The extraction zone then has a sufficiently large capture area while limiting head losses in the liquid flow.

The configuration of the chamber and particularly the face on which the supply orifice opens up are such that they give good liquid distribution in the chamber and therefore good contact between the liquid and the adsorption layer 7.

The chamber is supplied by means of a capillary 10 connected to the supply orifice 6 and the chamber is evacuated through a capillary 11 connected to the evacuation orifice 8. In the example shown and advantageously, the chamber is in the form of a hexagon with two larger dimension sides 12, parallel to the longitudinal axis.

Preferably, the supply orifice 6 and the outlet orifice 8 are located at two opposite vertices of the hexagon each connecting two sides 14, 16. The orifices 6 and 8 are then advantageously on the longitudinal axis X, so that good hydrodynamic performances can be obtained. Thus, the flow path is the longest possible in the chamber and the fluid distribution between the microstructures is improved by the tapered shape delimited by the sides 14.

The distance between the micropillars and the length of the chamber and the shapes of the micropillars are chosen to enable good extraction of analytes of interest contained in the sample and drying of the chamber. The chamber is preferably longer than it is wide to improve the distribution of liquid between the micropillars and improve the contact time between the liquid and the adsorbing phase. The ratio between the length and width of the chamber may for example be between 1 and 3.

For example, the extraction zone 4 may advantageously comprise heating means 18 shown diagrammatically, so as to enable desorption of the previously adsorbed analytes. For example, the heating means 18 are formed by one or several electrical resistances, for example located on an outside face of the module. Advantageously, means (not shown) are provided for measuring the temperature inside the adsorption area, for example consisting of two electrical resistances.

Operation of the device will now be described.

The extraction zone 4 is supplied with the liquid sample containing the analytes of interest via the capillary 10 and the supply orifice 6, the sample circulates inside the chamber between the micropillars. It comes into contact with the micropillars and the inside surface of the chamber and more particularly with the adsorption layer 7. The analytes are adsorbed by the adsorption layer 7. The sample is then evacuated through the outlet orifice. The flow in the chamber is adjusted to optimise the quantity of analytes adsorbed. It could also be envisaged to circulate the sample in the chamber again to increase the quantity of analytes adsorbed.

It could be envisaged to stop the flow of the sample in the chamber to improve contact, to slow it gradually until it stops or to maintain the flow through the chamber.

The inside of the chamber is the advantageously dried, for example by circulating a current of helium, dry air or nitrogen.

Therefore the chamber contains analytes adsorbed on the adsorption layer 7.

The analytes are desorbed during a subsequent step in order to be analysed, for example by a chromatography column associated with a detector. This is achieved by circulating a carrier gas in the chamber, for example helium, nitrogen or dry air. The heating means are then activated, which causes desorption of the analytes that are then carried by the carrier gas flow to the chromatography column or any other analysis device. In the case in which a chromatography column is used, the analytes are separated as a function of their affinity with the stationary layer covering the inside of the chromatography column and are detected for example by a detector at the outlet from the column.

The supply orifice 6 of the extraction zone 4 is advantageously connected to a carrier gas source and to a source of samples containing the analytes of interest through a valve with several flow channels. The outlet orifice from the extraction zone is advantageously connected to the detector and to a waste or collection zone through a valve with several flow channels. Valves may be switched under manual control or by automatic control.

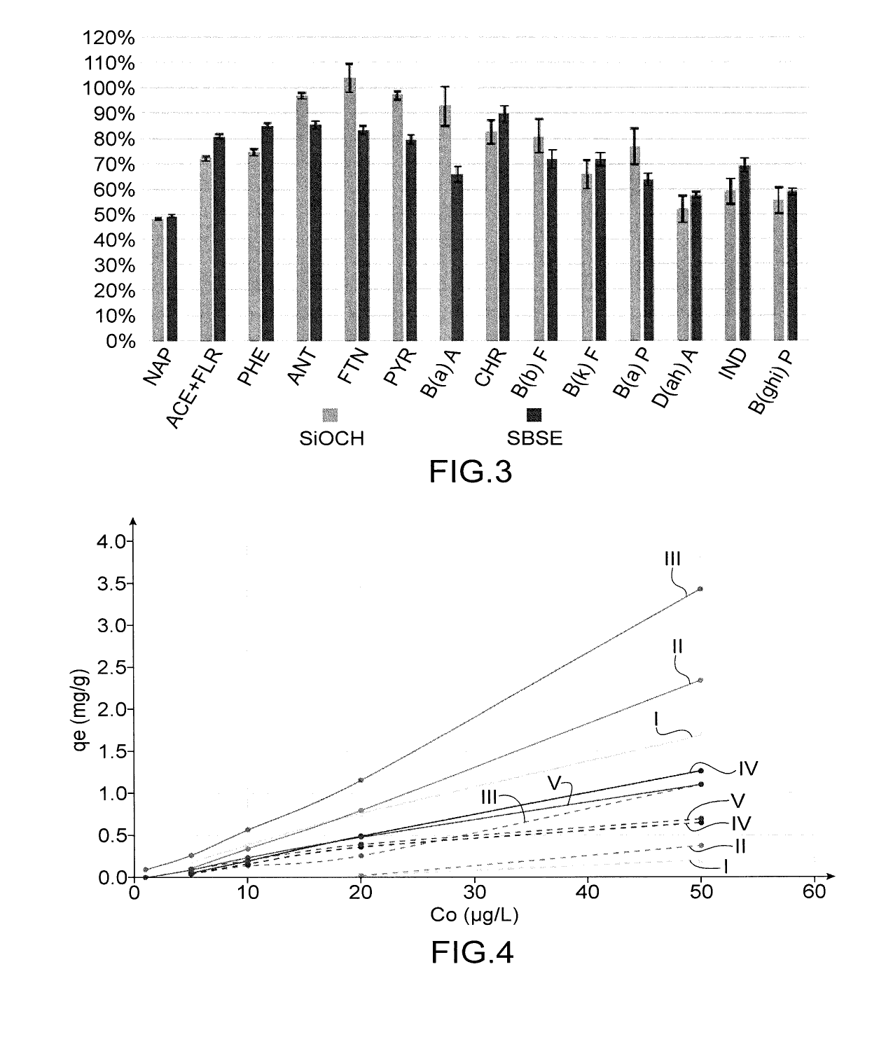

FIG. 3 contains a graphic view representing the extraction yield obtained by an extraction device according to the invention and using the SBSE technique with a magnetic bar coated with PDMS.

The extraction device comprises a chamber with a specific area of 14 cm². A 10 ml sample of water with a PAH content of 5 μg/l is analysed. Analytes were recovered by desorption using a solvent.

It is found that extraction yields obtained are comparable to yields obtained using the SBSE technique, with a bar having a length of 1 cm and coated with a 0.5 mm thick coat of PDMS, for the following PAHs:
Naphthalene (NAP),
Acenaphthene (ACE)+Fluorene (FLR),
Phenanthrene (PHE),
Anthracene (ANT),
Fluoranthene (FTN),
Pyrene (PYR),
Benzo(a)pyrene (B(a)A)
Chrysene (CHR),
Benzo(b)fluoranthene (B(b)F),
Benzo(k)fluoranthene (B(k)F),
Benzo(a)pyrene (B(a)P)
Dibenzo(a,h)anthracene (D(ah)A),
Indeno(1,2,3-c,d)pyrene (IND),
Benzo(ghi)perylene (B(ghi)P).

However, extraction by the device according to the invention is much faster than it is using the SBSE technique with a bar coated with PDMS, since the device according to the invention required about 20 minutes while about 24 hours was necessary using the SBSE technique. Therefore extraction with this invention is about 50 times faster than extraction using the SBSE technique with PDMS.

Furthermore, the extraction device shown in FIG. 1 is much more compact and more practical to use than the SBSE technique.

Therefore it is particularly suitable for use in the field. It is also easier to integrate into an analysis device.

FIG. 4 shows a graph of adsorption isotherms of different PAHs in which qe is the mass of different PAHs adsorbed by mass per phase in mg/g and Co is the initial concentration in the studied solution in μg/l, with an extraction device making use of a PDMS layer (curves in dashed lines) and with an extraction device according to the invention using an SiOxCyHz layer (curves in solid lines).

Curves I are for acenaphthene, curves II are for anthracene, curves III are for fluoranthene; curves IV are for benzo(a)pyrene and curves V are for indeno(1,2,3-c,d)pyrene).

In order to show that PAHs have better affinity towards SiOxCyHz than towards PDMS, 2.5×2.5 cm² silica slides functionalised with 150 nm of phase were immersed in a 10 ml of PAH solution stirred at 110 rpm for 24 h. Five concentrations were tested: 1 μg/l, 5 μg/l, 10 μg/l, 20 μg/l and 50 g/l. Desorption was done with 3 ml of acetonitrile for 20 minutes with ultrasounds. The solvent was then analysed by high performance liquid chromatography (HPLC), associated with fluorimetry detection (HPLC-FLD).

It is seen that the use of an SiOxCyHz extraction layer according to the invention can give 2 to 5 times higher extraction yields than an extraction device using PDMS. It is also seen that the factor between the two yields is higher when the compounds have low molar masses.

The following PAHs are classified by mass, this classification depending on ratios of yields with SiOxCyHz to yields with PDMS: acenaphthene>anthracene>fluoranthene>benzo(a)pyrene>indeno(1,2,3-c,d)pyrene).

Therefore the use of an SiOxCyHz extraction coat according to the invention can result in significantly higher extraction yields of compounds with 2-3 aromatic cycles, for example like acenaphthene, than those obtained by an extraction device using PDMS.

Porous silica extraction layers were tested for comparison, the affinity of these layers for PAHs is much lower than the SiOxCyHz extraction layer, for example this is the case for a mesoporous silica extraction layer formed by a sol-gel process with a porosity of 50%. This comparison also shows that the affinity of SiOxCyHz to PAHs is not related to its porosity alone, but to a particular chemical affinity.

This invention is particularly adapted to the extraction of analytes with aromatic cycles with an octanol/water coefficient (K_{ow}) of more than 10³. This applies particularly to persistent organic pollutants (POP) such as PAHs, HCB (hexachlorobenzene), PCBs (polychlorobiphenyls), PCDDs/Fs (Dioxins and Furanes), PBDEs (Polybromodiphenylethers) and organochlorine pesticides.

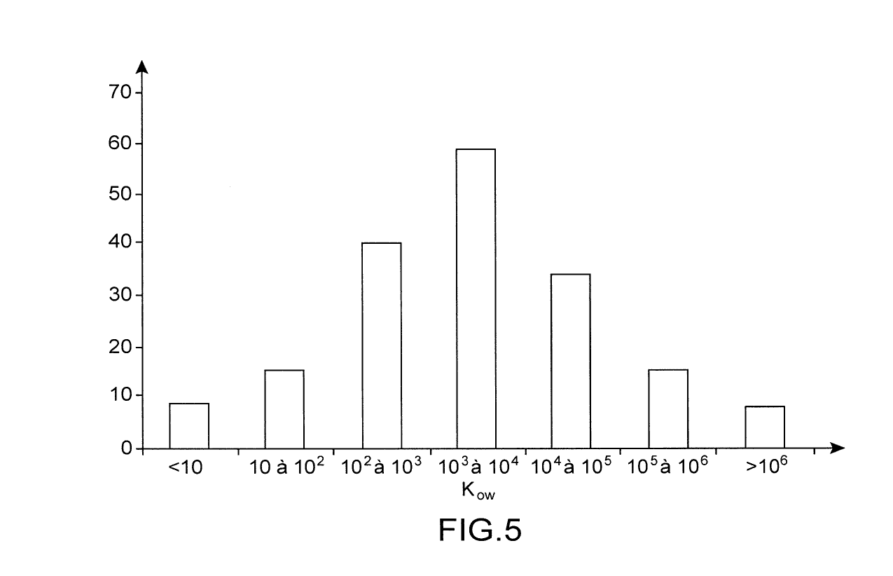

FIG. 5 shows the distribution of pesticides as a function of the value of K_{ow}, it can be seen that the coefficient K_{ow} for most pesticides is between 10² and 10⁵, and consequently the extraction device according to the invention is particularly efficient for the detection of a large number of pesticide families. Therefore this device can be envisaged for the extraction of pesticides with mean polarity (K_{ow} from 10³ to 10⁵) with little affinity for PDMS.

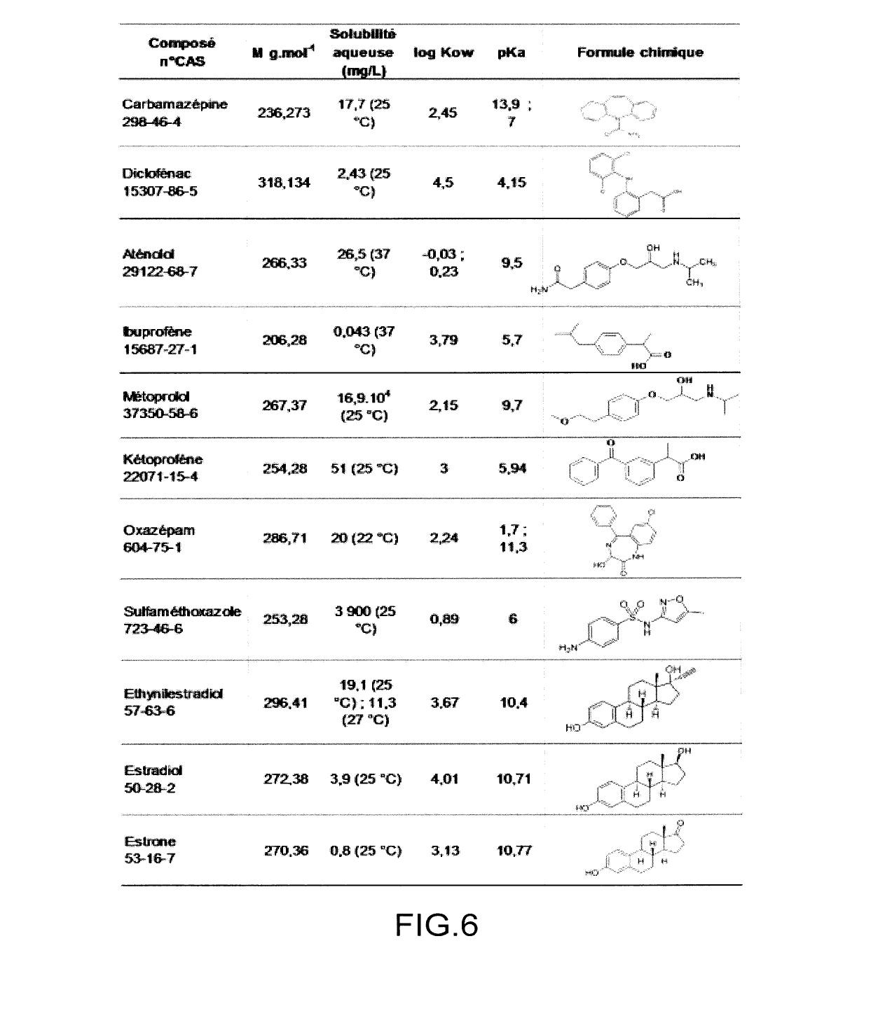

This invention is also advantageously applicable to the extraction of pharmaceutical substances for example such as diclofenac, ibuprofen, ketoprofen, ethinylestradiol, estradiol and estrone, that are aromatic molecules for which the value of log K_{ow} is between 3 and 4.5. FIG. 6 shows different pharmaceutical substances with their value of log K_{ow} including those listed above and that can be extracted by the device according to the invention.

An example of a method of making an extraction device according to FIG. 1 will now be described.

For example, the device may comprise a box provided with silicon micropillars and a glass cover. The box may be fabricated using classical techniques used in the field of micro-technologies.

A photolithography step can be applied to a substrate, followed by an etching step, advantageously by Deep Reactive Ion Etching (DRIE).

The SiOxCyHz adsorption layer is then formed on the etched surface of the substrate.

The SiOxCyHz layer can advantageously be deposited by Chemical Vapour Deposition (CVD). The SiOxCyHz is then conforming, i.e. its thickness is uniform along the entire length of the walls of the device. The addition of porogens during the deposition provides a means of controlling the porosity.

Even more advantageously, it can be deposited by Plasma-Enhanced Chemical Vapour Deposition (PECVD), by which the deposition can be made at low temperature to maintain the organic nature of the material. The uniformity of the spatial distribution of porogens and consequently pores formed during the elimination of porogens is improved.

For example, the SiOxCyHz adsorption layer is produced by plasma enhanced chemical vapour deposition starting from an organosilicate precursor and O₂. For example, TriMethylSilane (3MS), tetraMethylSilane (4MS), OctaMethylCycloTetraSiloxane (OMCTS) and DimethylDiMEthOxy Silane (DMDMOS) can be used as precursors. These materials have a structure composed of —Si—O—Si bonds in which some oxygen atoms are replaced by methyl groups.

Two precursors were used in one particular example: a diethoxymethylsilane organosilica matrix and an organic porogen, norbornadiene. Thus, a layer formed from a matrix containing organic inclusions of norbornadiene is obtained during the PECVD deposition. Organic inclusions are then eliminated by treatment, for example UV treatment and annealed at 400° C. The layer will then have some porosity.

According to another advantageous variant, the deposition may be made by Filament Assisted Chemical Vapour Deposition (FACVD), so that conformity can be optimised.

According to another variant, the deposition may be made by chemical vapour deposition of a first layer containing SiOxCyHz, with or without a porogen, followed by chemical vapour deposition of a second layer so as to form a second gas tight layer. The next step is a foaming step in which pores are formed in the SiOxCyHz layer. The second layer is then eliminated. For example, this example is described in patent U.S. Pat. No. 8,524,332. This method can obtain larger pores than are possible with CVD or enhanced CVD depositions.

As another variant, the SiOxCyHz can also be deposited using a sol-gel process, the porogen being initially mixed in the sol and then eliminated after gelification.

It would also be possible to apply a post-treatment to the SiOxCyHz layer to modify surface chemical conditions. Such a post-treatment can adjust the selectivity of the material for a given analyte. One example of a post-treatment is the application of an O₂, N₂ or N₂O plasma. Another example is silanisation, in other words covalent grafting of organic molecules using a silane function.

Variable thicknesses of SiOxCyHz are obtained as a function of the deposition duration.

For example, a thickness of 150 nm can be chosen, with a porosity of 30% measured by ellipso-porosimetry, the pores having an average radius of 1.3 nm.

Functionalisation by SiOxCyHz also has the advantage of providing better reproducibility of the extraction device than functionalisation by PDMS since the deposition of SiOxCyHz by PECVD can give a more conforming deposit and obtain controlled porosity.

During a next step, the cover is sealed onto the box, for example by anodic sealing.

Microfluidic connections are then made for example by capillaries. Inlets/outlets are along the centre-line of the chip.

A heating resistance, for example made of Pt, is made on the back face so that thermal desorption of the adsorbed analytes is possible. To achieve this, an electrical insulating layer is formed beforehand, for example made of SiO₂, on the back face of the substrate and an electrical conducting layer, for example made of a platinum layer, is then formed on the oxide layer. The platinum layer is then structured, for example by photolithography and etching. Advantageously, means are made to measure the temperature of the chip, for example by making two resistances integrated on the back face.

This invention also relates to any device for extraction of analytes of interest with aromatic cycles, preferably analytes with aromatic cycles for which the octanol-water partition coefficient is more than 10³, contained in a liquid phase, using an SiOxCyHz adsorption layer, non-limitatively such as devices using SBSE, SPE and SPME techniques, providing improved extraction yields with a shorter analysis time.

1. The extraction device for the extraction of analytes with aromatic cycles, said analytes being contained in a liquid phase, said extraction device comprising a support and an adsorption layer being porous SiOxCyHz, said adsorption layer at least partially covering said support, the extraction device being configured to put into contact the liquid phase and the adsorption layer.

2. The extraction device according to claim 1, in which the octanol-water partition coefficient of analytes of interest with aromatic cycles is more than 10³, and advantageously between 10³ and 10⁵.

3. The extraction device according to claim 1, wherein x is between 1 and 2 and preferably between 1.4 and 1.8; y is between 0.8 and 3 and preferably between 1 and 2.5 and z is between 2.5 and 4.5 and preferably between 3 and 4.1.

4. The extraction device according to claim 1, wherein the thickness of the adsorption layer is between 50 nm and 2000 nm, and preferably between 50 nm and 1000 nm.

5. The extraction device according to claim 1, wherein the porosity of the adsorption layer is between 3% and 60%, and preferably between 10% and 40% and the radius of pores is between 1 nm and 5 nm.

6. The extraction device according to claim 1, wherein the support is composed of at least the walls of an extraction chamber, said extraction chamber comprising at least one liquid phase supply orifice and at least one liquid phase outlet orifice, said supply and outlet orifices being arranged such that the liquid phase comes into contact with the adsorption layer and flows from the supply orifice towards the extraction orifice.

7. The extraction device according to claim 6, wherein the extraction chamber comprises microstructures at least partly coated by the adsorption layer composed of porous SiOxCyHz.

8. The extraction device according to claim 7, wherein the microstructures are micropillars supporting the absorption layer.

9. The extraction device according to claim 6, including a box containing the chamber, and a cover closing the chamber.

10. An analysis system comprising an extraction device according to claim 1 and a device for the analysis of compounds extracted by the extraction device.

11. The analysis system according to claim 10, wherein the analysis device is a chromatography column associated with a detector, advantageously a chromatography microcolumn associated with a detector.

12. A method of manufacturing an extraction device according to claim 1, including the following steps:
a) make a support,
b) form a layer of SiOxCyHz on the support,

13. A manufacturing method according to claim 12, wherein step b) is done by chemical vapour deposition, advantageously plasma-enhanced chemical vapour deposition.

14. The manufacturing method according to claim 12, wherein a porogen, for example norbornadiene, is used in step b) during formation of the SiOxCyHz layer, with an annealing step to eliminate the porogen.

15. The manufacturing method according to claim 12, wherein the support is formed by the internal walls of an extraction chamber, said chamber being made by photolithography and etching, advantageously DRIE etching.

16. A method for extraction of compounds with aromatic cycles that advantageously have octanol/water partition coefficients of more than 10³ contained in a liquid phase, making use of an extraction device according to claim 1, including the following steps:
bring a liquid sample comprising at least one aromatic compound advantageously having an octanol/water partition coefficient of more than 10³ into contact with the absorption layer.
